(19) United States
(12) Patent Application Publication
(10) Pub. No.: US 2005/0082175 A1
(43) Pub. Date: Apr. 21, 2005

(54) Diagnosis by sensing volatile components

(75) Inventors: Selwayan Saini, Millbrook (GB); Conrad Bessant, Bedforshire (GB); Christian Liedtke, Leonberg (DE); Jan Leiferkus, Oxford (GB)

Correspondence Address: OBLON, SPIVAK, MCCLELLAND, MAIER & NEUSTADT, P.C., 1940 DUKE STREET, ALEXANDRIA, VA, 22314, US

(73) Assignee: Cranfield University, Bedfordshire (GB)

(21) Appl. No.: 10/475,310

(22) PCT Filed: Apr. 19, 2002
(86) PCT No.: PCT/GB02/01853

(30) Foreign Application Priority Data
Apr. 19, 2001 (GB) 0109572.8

(51) Int. Cl. G06F 19/00; G01N 31/00; C12Q 1/00; G01F 1/64

(52) U.S. Cl. 205/777.5; 702/22

(57) ABSTRACT

A state, particularly a disease state, associated with the production of volatiles is detected by passing a sample containing the volatiles to a single sensor. This may be a semiconductor gas sensor element or a surface acoustic wave device. This provides an output signal, e.g. in the form of a tailing peak. A plurality of characteristics of the signal (e.g. peak height and maximum positive gradient) are measured to characterise the sample and hence the underlying state. For example we can discriminate between urine samples which are (a) infected with proteus, (b) infected with E. coli or (c) uninfected.

### TECHNICAL FIELD

This invention describes a method and apparatus for detecting volatile components to deduce the state of a system involved in their production. The system may comprise a patient's body, or part thereof. Thus a detector assembly can be used to detect the odour emissions from diseases caused by micro-organisms or from metabolic changes brought about through disease.

### BACKGROUND ART

The smell associated with bacterial infection and the putrefaction associated with tissue destruction have long been recognised. It is now clear that many bacterial species produce specific odours. This is most notable in the anaerobic bacteria such as bacteroides and clostridia species. Often a pungent odour can only be detected if there is an overwhelming infection. Given a mild infection in the urine of a child with Eschericha coli imparts a distinct fishy odour, often detected by the mother who is familiar with the normal smell.

Certain metabolic diseases may be first detected from the patient's odour. Prior to the advent of molecular and biochemical medicine, the sweet smell imparted to the breath of an unconscious child was the first clue of an acidotic diabetic coma. A musty odour in an older patient could indicate imminent hepatic failure, or a urinose smell, kidney failure. Halitosis or simple bad breath may have many causes, but is often related to excessive bacterial overgrowth in the stomach (associated with gastric cancer) or in the lungs (with bronchiectasis and secondary infection).

The sense of smell has long been used as a means of disease diagnosis. Indeed, many diseases are known to emit characteristic odours that are used as markers of the diseased state (Barr et al., 2001).

Detection of Microbial Odours

The rapid detection of an infecting organism can be extraordinarily helpful for the correct treatment of patients. The result of bacterial infection is often to produce pus, which is a mixture of bacteria and dead white blood cells. Pus can easily be seen but the causal bacteria have often to await culture techniques to allow detection. Rapid detection allows correct antibiotic treatment to be given. The rapid detection of microbes is not only important in medicinal practice but also in the food and drink industry and in environmental monitoring. A number of “rapid” methods have been described based on different chemical, physical and biological techniques (Hobson et al., 1996).

It is well known that different microbes display different metabolic pathways. These biochemical differences are inherent to the microbe and can be utilised as a means of detecting different microbial species. A common way of doing this is by the use of selective nutrient media that promote the growth of certain microbes by making available nutrients that can be metabolised for growth leading to the formation of visible colonies on a culture plate. Such microbiological plating techniques are widely used for microbial identification purposes. An extension of these ideas is the detection of metabolic volatile products that emanate from specific micro-organisms. A good example of this approach is embodied in research performed as far back as the 1970s where complex laboratory analytical techniques were used to identify specific end-products of metabolism as a means of identifying different microorganisms. Reports by Hayward et al. in 1977 and by Coloe in 1978 describe the detection of metabolic volatile end-products from E. coli and P. mirabilis using gas-liquid chromatography. In this work, the metabolic activity of bacteria on growth media led to the production of volatile chemicals that appeared in the head space of the growth vessel and were subsequently detected using a gas-liquid chromatography detector. Since different microbes display different metabolic pathways, it became feasible to distinguish between different species by recognising the formation of specific volatile markers using the gas-liquid chromatography detection method. The work of Hayward et al. in 1977 and Coloe in 1978 showed this approach to be highly effective in the identification of E.coli and P. mirabilis. These authors successfully applied the microbial odour analysis method to the rapid diagnosis of bacteria responsible for urinary tract infections using the chromatography detector.

Further work in the 1980s and 1990s utilised a different detector for microbial volatile products using an array of chemical sensors. These array-based detectors for volatile chemicals employed a number of broad-specificity gas sensors. Such detector assemblies are commonly known as electronic noses. In this respect, WO 97/ 08337 and U.S. Pat. No. 5,807,701 describe methods for the identification of microbes using arrays of sensors that respond to the different gases or vapours that are produced by different microbes grown in nutrient media. Since different microbial species display different metabolic products, a broadly responsive array is thought to provide a good detector in order to capture sufficient information to make subsequent predictions on which species are present more accurate. The sensors in the array interact with the different products causing multiple sensor signals that are subsequently collectively analysed by pattern recognition techniques using software. By using an appropriate pattern recognition technique, it becomes possible to recognise sensor patterns produced by different microbes.

Detectors of Odours from Metabolic Diseases

A number of different techniques have been employed to examine the chemical make-up of odours from bodily fluids such as those imparted to breath and urine. These detection techniques are broadly similar to those already described for the detection of microbial odours, namely, complex instrumentation such as gas chromatographs and mass spectrometers and the electronic nose. Recently, the use of odours for metabolic disease detection was shown: The combination of twenty-two breath volatiles (predominantly alkanes, their derivatives and benzene compounds) could be used to discriminate between patients with and without lung cancer (Phillips et al., 1999) creating the possibility of a lung cancer ‘breathalyser’ for rapid diagnosis and screening (Gardner and Bartlett, 1994).

### DISCLOSURE OF INVENTION

In one aspect the invention provides a method of testing for the occurrence of a state associated with the generation of volatile components comprising:
a) providing a gaseous sample potentially containing said volatile components;
b) exposing a single sensor device to said sample, said sensor device being capable of generating an output signal in response to a range of different volatile components;
c) determining a plurality of parameters of the output signal, and correlating the plurality of determined parameters with predetermined parameter patterns associated with one or more states whose occurrence is to be detected.

There may be a calibration step in which a range of known samples are tested to establish boundaries in an n-dimensional space, where n is the number of parameters used, delimiting the response patterns associated with one or more states. Then in a test using an unknown, if the result falls within the boundary associated with one or said states, this is deemed to indicate the existence of that state in the sample's source.

In a second aspect the invention provides apparatus for carrying out such a method, comprising a sensor device, a gaseous sample supply system and a computing device (e.g. a computer or microcontroller), suitably connected together.

A preferred embodiment is concerned with a new detector assembly designed to detect disease odours from infecting micro-organisms or metabolic disease odours imparted to bodily fluids. The disease detection capability of the invention is illustrated in the sections below. It is shown how the assembly can be used to detect disease odours from urine samples provided by patients suffering from urinary tract infections. The new detector assembly uses a single gas sensor making it significantly different to the electronic nose devices that incorporate multiple array sensors as described in WO 97/ 08337 and U.S. Pat. No. 5,807,701. Furthermore, the detector assembly is different to previously described gas-liquid chromatography and mass spectrometer detectors that have been used. Since only a single sensor element is used for monitoring disease odours, the detector assembly offers a number of important possibilities in the detection of disease odours for medical diagnostics applications.

### BRIEF DESCRIPTION OF DRAWINGS

FIG. 1 is a schematic view of a sensor assembly embodying the invention.

FIG. 2 is an example of a sensor response curve.

FIG. 3 is a graphic display showing bacterial discrimination from maximum positive slope and peak height (normalised and mean centred).

### MODES FOR CARRYING OUT THE INVENTION

Detector Assembly

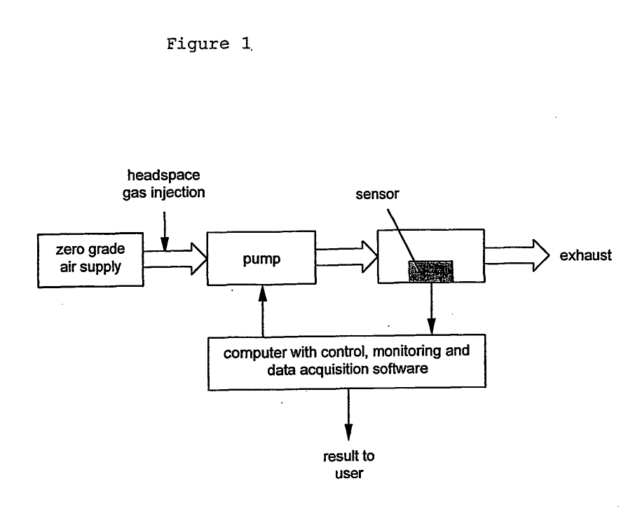

The detector assembly comprises a gas sensor, a means of delivering disease odour samples to the sensor and an electronic interface controlled via a personal computer. The detector is shown schematically in FIG. 1. The single gas sensor used in the detector can be of a variety of materials whose properties change in the presence of disease odours. The response generated therein is recorded by the electronic interface connected to a computer or programmable micro-controller circuit. In contrast to electronic nose detectors, the signal obtained from the detector is from one sensor only as opposed to multiple sensors. By simultaneously considering several aspects of the single sensor response, it is possible to determine if the sample contains disease odours.

In one embodiment, a metal oxide semi-conductor (MOS) sensor is used. MOS sensors have been widely employed for several gas detection applications and a variety of sensors are available commercially. MOS sensors are also widely used in array based electronic nose devices. Gas sensors employing semi-conducting oxides generally operate at temperatures above ambient. The electrical resistance of the sensor material depends upon the temperature, and also on the chemical composition of the surrounding atmosphere. It has been known for many years that, when heated, most oxides change their resistance as the oxygen concentration in the atmosphere changes. Most of the semi-conducting materials used in gas sensors share a common dependence of their resistance on the concentration of the target gas in the surrounding atmosphere. For most gases except oxygen, the change in resistance per unit change in gas concentration is greatest at lower concentrations of the target gas, and it decreases as the concentration of the target gas increases. Mathematically, this behaviour can generally be fitted either to a logarithmic curve, or one which varies as the square root of the concentration of the target gas.

The nature of the response from MOS sensors is dependent on the type of interaction volatile compounds present in the sample undergo with this form of sensor, as well as other measurement parameters. Metal oxide materials are polycrystalline and can be either p-type or n-type semiconductors and for odour detection applications are operated at elevated temperatures (300-550° C.). Oxygen in the purge gas passes over the sensor surface and reacts reversibly with lattice vacancies in the metal oxide to produce various oxygen species (O₂, O⁻, O₂⁻). This process extracts electrons from the bulk, which in the case of n-type materials such as SnO₂ as used here, reduces the charge carriers and hence increases the measured resistance. Exposure of the MOS film to reducing analytes will allow certain species to react with the oxygen ions forming products which may either de-sorb or generate other surface species. In general such processes will release electrons back to the sensor material, which decreases the resistance of an n-type MOS sensor. It is this change in resistance that is measured to generate the sensor response.

A further type of sensor material that can be used in the detector assembly is an organic semi-conducting polymer. In this sensor, a polymer based on poly-pyrole or poly-aniline is electrochemically polymerised across the surface of two or more electrode elements. The resistance in the polymer changes in response to the composition of the adjacent gas sample. Such sensors have also been widely used in array configurations in electronic nose detectors.

It is also possible to use an acoustic wave device. This may be modified by the presence of a chemical layer that forms the sensing surface.

Surface Acoustic Wave (SAW) devices use thin film interdigital metal electrodes fabricated on piezoelectric substrates both to generate and to detect surface acoustic waves. Surface acoustic waves are waves that have their maximum amplitude at the surface and whose energy is nearly all contained within 15 to 20 wavelengths of the surface. These waves can exist where a solid material has a free bounded surface. Because the amplitude is a maximum at the surface such devices are very surface sensitive. Normally SAW devices are packaged in hermetically sealed cavity style packages to ensure that their performance will not change owing to a substance contacting the surface of the SAW die.

SAW chemical sensors take advantage of this surface sensitivity to function as sensors. If a SAW device is coated with a very thin polymer film it will affect the frequency and insertion loss of the device. If the device with the polymer coating is then subjected to chemical vapours that will adsorb into the polymer material, then the frequency and insertion loss of the device will further change. It is this final change that allows the device to function as a chemical sensor.

The polymer film is normally chosen so that it will have a chemical affinity for a variety of organic chemical classes. An additional SAW device with no polymer film may also be used as a reference for the coated device.

The sensitivity of the system can be enhanced for low vapour concentrations by having the option of using a chemical concentrator before the array. In operation, the concentrator adsorbs the test vapours for a period of time and is then heated to release the vapours over a much shorter time span thereby increasing the effective concentration of the vapour at the array.

Technical Section

In order to demonstrate the invention, the detector is used to monitor disease odours present on urine samples collected from patients suffering urinary tract infections. Infections of the urinary tract such as cystitis are very common, affecting as many as 20% of women under the age of 30. Women are particularly susceptible because of the shorter length of the female urethra making the ascent of exogenous bacteria more likely than in males. Left unchecked, an infection can spread to the kidneys, causing a number of complications including kidney damage. Urinary tract infections (UTIs) tend to impart unusual odours to the patient's urine that are caused by volatile by-products of the infecting microbe. UTIs therefore provide an ideal opportunity to evaluate the new detector assembly for disease odours.

Current UTI diagnosis involves urine culture: the growth of bacteria on selective media, which leads to the identification of the responsible organism. This test takes a minimum of 24 hours although 48+ hours is usual owing to the large number of samples that need to be processed in a busy hospital microbiology laboratory. This can be in addition to the time elapsed before the sample is received from a physician's surgery following initial presentation. Once urine culture has identified the offending organism, the results are returned to the referring doctor who will normally prescribe a course of antibiotics. The main clinical advantages of the detector assembly for UTI diagnosis is the potential for reducing the time required in identifying the organism responsible for the infection by performing the analysis at the point of initial presentation (doctors office) or within a hospital laboratory.

Technical Description

Preparation of Clinical Urine Samples

Urine samples from patients suffering from UTIs were obtained from a local hospital that has confirmed the presence of bacterial infection using plating methods. Two types of clinical urine sample were provided—one infected with E. coli and the other by Proteus mirabilis. A third sample set were urine samples obtained from healthy individuals. For analysis, the bacterial cells from 0.5-5 ml volumes of each sample were collected, resuspended in bovine heart infusion (BHI) broth supplemented with 0.1 M L-methionine and 1% (w/v) lactose, and cultured for 2-4 hours as follows: a 5 ml volume of sample was taken up in a 5 ml syringe and filtered through a Minisart device containing a 0.2 μm cellulose acetate membrane (Sartorius) until the filter blocked. A No. 16 gauge (0.6 mm) needle was attached to the outlet of the Minisart device and cells collected on the membrane resuspended in 5 ml BHI by backflushing with sterile media taken from a 20 ml glass headspace vial. By removal of the Minisart filter and re-attachment of the needle to the syringe, resuspended bacteria (in a total volume of 5 ml) were injected back into the vial. Samples were incubated at 37° C. without agitation until the optical density (OD) of the culture at 600 nm was 0.7. To avoid disturbing cultures for analysis, duplicate samples were set up for OD determinations. As a control, a selection of uninfected samples were acquired from members of laboratory, and underwent the sample process as the infected samples.

Detection of Disease Odours using Detector Assembly

At the end of the incubation period, samples were removed from the incubator, and vortexed for 5 seconds. A 5 cm³ sample of headspace gas was removed from each vial using a Hamilton gas syringe and immediately injected into a stream of zero grade air, which carried the sample across a single metal oxide sensor at a flow rate of 50 ml/min a pump (FIG. 1 shows the detector arrangement). The metal oxide sensor was obtained from a local manufacturer and operated at a constant elevated temperature as is the normal mode of operation with these devices.

The resistance of the metal oxide sensor was monitored once per second over a two minute period. Prior to the first measurement, the sensor was allowed to stabilise by passing air over it at a rate of 50 ml/min for five minutes. After each measurement, the sensor was cleaned by passing air over for 10 minutes at a rate of at 200 ml/min. Samples were analysed in a randomised order over a series of days, to decouple the effects of any sensor drift from the genuine differences between samples.

Data Analysis

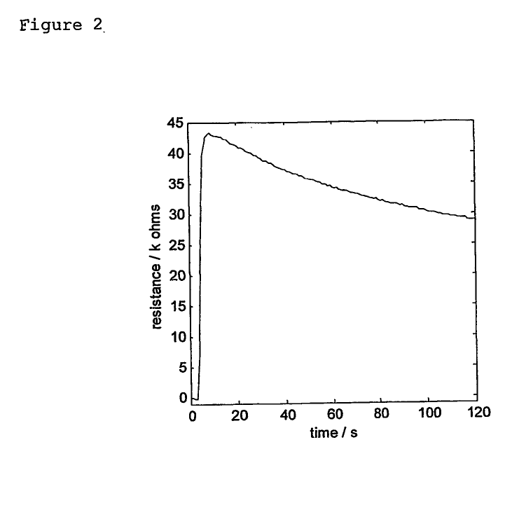

When sensor resistance is plotted as a function of time, it takes the form of a tailing peak (FIG. 2). The following characteristic parameters were determined from the sensor response:
Peak height
Maximum positive gradient
Maximum negative gradient
Time at which peak occurs
Response decay constant
Logarithmic slope

These were then combined into an i×j data matrix X_{i,j}, where i is the number of samples analysed and j is the number of parameters of interest. For pairs of parameters, this matrix is easily visualised by plotting one parameter against the other. For more parameters, principal components analysis (PCA) can be used to reduce the data to two dimensions.

The data pre-processing methods of normalisation and mean centring were employed to maximise the discrimination seen in PCA. These methods are described below.

No malisation: For each sample, the parameters acquired from the sensor are scaled such that the summed response for each sample is constant for all samples. This removes any gross changes in response from sample to sample, so can be useful in overcoming instrumental drift and variations in the strength of infection.

$${}_{}^{n}{x}{}_{ij}^{} = \frac{{x}_{ij}}{{\sum }_{j = 1}^{J} {x}_{ij}}$$

Mean centring: The mean average value of each parameter over all samples is subtracted from all the measurements for that sensor. This removes any residual features from the data so, for example, if one sensor parameter always produces a large response, this does not dominate over other sensors.
^{mc}x_{if}=x_{if}−\overline{x}ⱼ
Results

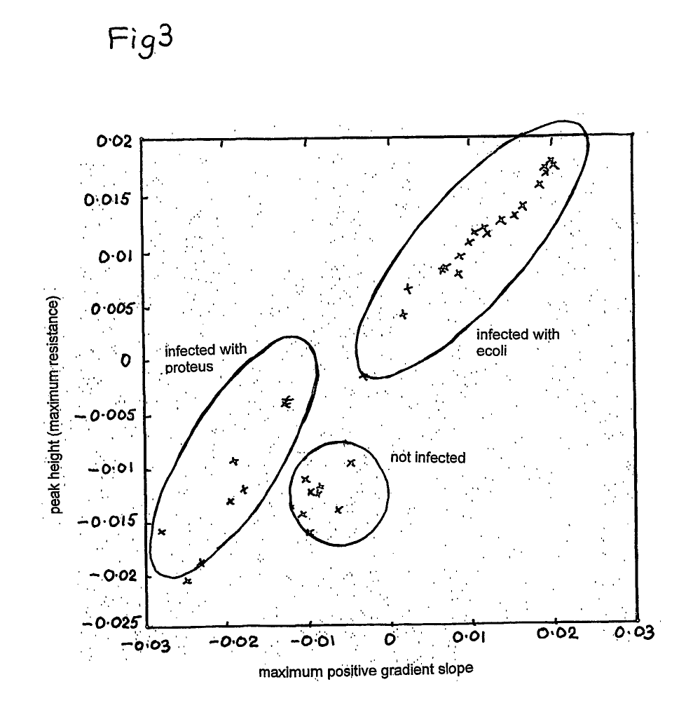

Two parameters (peak height and maximum positive gradient) were found to provide enough information to successfully resolve different sample types for clinical samples incubated for 2-4 hours (see FIG. 3). FIG. 3 shows the results. Urine samples obtained from patients infected with either proteus or E.coli form separate clusters in the plot. The odour detector successfully detected each of the disease causing bacteria compared to urine samples obtained from individuals with no UTI. Furthermore, the overall time for diagnosis has been markedly reduced from the usual 24 hours required with plating methods.

Other Features of Detector Assembly

The example shown in the technical description is of clear benefit to clinical diagnostics, which forms an ideal application of the new detector assembly. The detector also offers a number of other significant advantages compared to the electronic nose or complex laboratory detectors. Advantageous features for disease detection in medical diagnostics include:
1. Since only a single sensor is required in the detector, the overall size of the detector can be markedly smaller (typically a few cm in the largest dimension) than an electronic nose or chromatography detectors. Small detector size may allow it to be used in-vivo or as part of an endoscope or bronchoscope for internal investigations.
2. A single sensor detector has lower power consumption compared to arrays used in electronic nose detectors and chromatography instruments. Lower power and smaller size dimensions suggest the use of mobile detectors or as part of mobile devices.
3. The quantitative nature of the detector makes it possible to measure the concentration of disease odours present in a sample which could be useful in the application of drug dosage regimes in patient treatment.
4. Since only one sensor is used, the computer power required is vastly reduced.
5. The overall cost of the detector is orders of magnitude less than the cost of commercial electronic nose arrays and laboratory chromatography equipment. The possibility for low cost, disposable devices is evident for mass markets such as home testing.
   Cited Literature
Barr, H., Bessant, C., Saini, S. Sniffing out disease using the artificial nose. Journal of the Institute of Biology, 148 (5), 229-233 (2001).
Coloe et al. Journal of Clinical Pathology (1978). vol 31, pp 361-364.
Gardner, J. W., Bartlett, P. N. (1994). A brief history of electronic noses. Sensors and Actuators B-Chemical 18, 211-220.
Hayward N J et al. Journal of Clinical Microbiology (1977). vol. 6, September 1977, pp 195-201.
Hobson, N. S.; Tothill., I., Turner, A. P. F. (1996). Biosensors and Bioelectronics, vol 11, No. 5, pp 455-477.
Phillips, M., Gleeson, K., Hughes, J. M. B., Greenberg, J., Cataneo, R. N., Baker L., -McVay, W. P. (1999). Volatile organic compounds in breath as markers of lung cancer: a cross-sectional study. Lancet 353, 1930-1933.
   Patents Cited

1. Patent Number WO 97/08337, Publication Date 6 Mar. 1997.

2. Patent Number U.S. Pat. No. 5,807,701, Publication Date: Sep. 15, 1998.

1. A method of testing for the occurrence of a state associated with the generation of volatile components comprising:
a) providing a gaseous sample potentially containing said volatile components;
b) exposing a single sensor device to said sample, said sensor device being capable of generating an output signal in response to a range of different volatile components;
c) determining a plurality of parameters of the output signal, and correlating the plurality of determined parameters with predetermined parameter patterns associated with one or more states whose occurrence is to be detected.

2. A method according to claim 1 wherein the sensor device is an electrical device having a sensor element whose electrical resistance is affected by exposure to volatile components.

3. A method according to claim 1 wherein said sensor is an electrical device having a semiconductor sensor element.

4. A method according to claim 3 wherein said element is a metal oxide semiconductor (MOS) element.

5. A method according to claim 3 wherein said element is an organic semiconducting polymer element.

6. A method according to claim 5 wherein said element is based on polypyrrole or polyaniline.

7. A method according to claim 1 wherein said sensor is a surface acoustic wave device that is responsive to volatiles in the sample.

8. A method according to claim 7 wherein said acoustic wave device is modified with a chemical layer that forms the sensor element.

9. A method according to claim 1 wherein the state to be detected is a disease state.

10. A method according to claim 9 wherein the disease state affects at least one of: the urinary tract; the gastrointestinal tract; the respiratory system; soft tissues; skin; auditory and olfactory system; circulatory system; and the central nervous system.

11. A method according to claim 1 wherein the volatile components are generated by microorganisms, and step (a) includes a first step of obtaining a sample of material potentially containing the microorganisms and culturing it under conditions such that the microorganisms would generate the components.

12. A method according to claim 1 wherein the volatile components are generated by body processes, and step (a) includes a first step of obtaining a sample of material potentially containing the volatiles of interest.

13. A method according to claim 1 wherein step (a) includes a pre-concentration step to increase the concentration of the volatiles before presenting to the sensor device.

14. A method according to claim 1 wherein exposure of the sensor device to the sample leads to an output signal in the form of tailing peak, and in step (c) the parameters are selected from (i) peak height, (ii) maximum positive gradient, (iii) maximum negative gradient, (iv) time at which peak occurs after exposure of the sensor to the sample; (v) response decay constant; and (vi) logarithmic slope.

15. A method according to claim 14 in which the parameters are (i) and (ii).

16. A method according to claim 1 in which the magnitudes of one or more of said parameters are used to provide quantitative data about the amounts of volatile components.

17. Apparatus for use in carrying out the method of claim 1 including said single sensor device, a gaseous sample supply system coupled to the sensor device for providing samples to the sensor device, and a computer or microcontroller coupled to the sensor device for receiving and analysing its output and providing a display and/or an output signal indicative of the result.

18. Apparatus according to claim 17 wherein the sensor device is part of an endoscope or bronchoscope.
